(19) United States
(12) Patent Application Publication
(10) Pub. No.: US 2011/0311657 A1
(43) Pub. Date: Dec. 22, 2011

(54) Use of a Neuroprotective Extract and Pharmaceutical Preparation Thereof

(75) Inventors: Suhaila Mohamed, Kajang (MY); Juliana MD Jaffry, Kuantan (MY); Intan-Natasya Ahmad, Alor Star (MY)

(73) Assignee: Universiti Putra Malaysia, UPM Serdang (MY)

(21) Appl. No.: 13/203,803

(22) PCT Filed: Dec. 2, 2009
(86) PCT No.: PCT/MY09/00202

§ 371 Date: Aug. 29, 2011

(30) Foreign Application Priority Data
Mar. 11, 2009 (MY) PI 20090964

(51) Int. Cl. A61K 36/889; A61P 39/06; A61P 25/00

(52) U.S. Cl. 424/727

(57) ABSTRACT

The present invention discloses an extract having neuroprotective property prepared from a plant of the family Palmae, which is capable in preserving neurons against damage caused by oxidation, ageing and/or nitric oxide deficiency. Also disclosed is a pharmaceutical preparation and a comestible preparation comprising the extract as an active ingredient in a therapeutically effective amount to protect against neuron damage.

The present invention relates to an extract having neuroprotective property. More particularly, the present invention relates to an extract from palm leaves which is capable of protecting against neuron damage caused by oxidative stress, nitric oxide deficiency and/or ageing.

### BACKGROUND TO THE INVENTION

One important issue in brain aging is the spectrum of memory and cognitive loss that occurs from normal aging. The brain accounts for only a few percent of the body weight but it processes 20% of basal oxygen (O₂) consumption. Under physiological O₂ level, 1% to 2% of the O₂ consumed is converted to reactive oxygen species (ROS). Besides, it is enriched with readily peroxidizable polyunsaturated fatty acids. The brain is not highly equipped with antioxidant defenses as it has a very low level of catalase activity and only moderate amounts of the endogenous antioxidant enzymes, superoxide dismutase and glutathione peroxidase. Oxidative stress, mitochondrial dysfunction, and apoptosis are involved in basic molecular and biological processes leading to neuronal cell death.

Much of the work elucidating the relationship between reactive oxygen species (ROS) and aging, and nitric oxide and neuron signaling have focused on hippocampal function, because the decreased hippocampal function is well known to be correlated with biomarkers of aging and is associated with a number of neurodegenerative disorders, such as Alzheimer's disease. Nitric oxide (NO) is an organic gas that plays a role in the control of cerebral blood flow, thrombogenesis, and modulation of neuronal activity. In the brain, nitric oxide (NO) is produced by endothelial cells, neurons, glia, and macrophages by three different isoforms of the enzyme nitric oxide synthase (NOS). Nitric oxide synthase from endothelial cells (eNOS) and neurons (nNOS) are both constitutively expressed enzymes, whose activities are stimulated by an increase in intracellular calcium. The third isoform is high-output inducible nitric oxide synthase (iNOS) expressed during inflammatory reactions and mediates cytotoxicity in many cell systems including central nervous system (CNS). The localization of a neuron-specific nitric oxide synthase indicates that nitric oxide has a widespread action in the central nervous system (CNS). The highest levels of nitric oxide (NO) throughout the body are found also in neurons, where nitric oxide (NO) functions as a unique messenger molecule, modulating neuronal activity.

Behavioural studies have revealed nitric oxide (NO) involvement in certain forms of memory formation. The systemic administration of nitric oxide synthase inhibitors such as L-NAME has been shown to impair spatial learning in rats in both a radial arm maze and in Morris water maze tasks, and in a step-down inhibitory avoidance task. Nitric oxide (NO) memory storage depends on nitric oxide-sensitive processes in the hippocampus.

Metabolites from a limited number of plants have been proven to possess neuroprotective properties. A few neuroprotective products containing plant metabolites as the active ingredients have been disclosed.

United States publication no. 20070060644 by Vander Jagt et al, discloses a method of treating a subject afflicted with Alzheimer's disease or type 2 diabetes. The method comprising administering to the subject a therapeutically effective amount of a composition comprising a curcumin derivative selected from the croup consisting of: (a) Ar¹-L-Ar² (Formula I); wherein L is a divalent linking group comprising an alkylene or an alkenylene comprising 3, 4, 5, 6, or 7 backbone carbon atoms, wherein one or more of the backbone carbon atoms form part of a carbonyl or secondary alcohol; and Ar¹ and Ar² are each independently aryl groups; and (b) Ar¹-L-R¹¹ (Formula IV); wherein L is a divalent linking group comprising an alkylene or an alkenylene comprising 3, 4, 5, 6, or 7 backbone carbon atoms, wherein one or more of the backbone carbon atoms form part of a carbonyl or secondary alcohol; Ar¹ is an aryl group; and R¹¹ is an alkyl group, a heterocyclic group, or a hydrogen.

U.S. Pat. No. 6,939,570 to Snow et al., discloses a pharmaceutical agent for treating an amyloid disease such as Alzheimer's Disease that includes a therapeutically effective amount of an extract obtained from the inner bark or root tissue of a plant of the genus Uncaria, species tomentosa, wherein the weight percentage of plant extract in the agent is in the range of from about 70% to about 95%.

U.S. Pat. No. 6,607,758 to Castillo and Snow discloses a method of inhibiting amyloid formation, deposition, accumulation, or persistence, or amyloid protein-amyloid protein interactions, amyloid-proteoglycan interactions, amyloid-PG/GAG interactions and/or amyloid-glycosaminoglycan interactions, and/or dissolving or disrupting pre-formed or pre-deposited amyloid fibrils in Alzheimer's Disease in a mammalian subject. In the method a therapeutically effective amount of plant matter from the genus Uncaria, species tomentosa is administered, preferably from the inner bark or root tissue of Uncaria tomentosa.

U.S. Pat. No. 6,264,994 to Castillo et al., discloses a composition of plant matter comprising Uncaria tomentosa and at least one of ginkgo biloba, rosemary, golu kola and bacopin. Use of extracts from the inner bark and root parts of Uncaria tomentosa, and use of the ingredients contained within the various commercial preparations of Uncaria tomentosa, benefit human patients with Alzheimer's disease and other amyloidoses due to Uncaria tomentosa's newly discovered ability to inhibit amyloid fibril formation, inhibit amyloid fibril growth, inhibit amyloid-proteoglycan interactions, inhibit amyloid-glycosaminoglycan interactions, and cause dissolution and/or disruption of preformed amyloid fibrils.

U.S. Pat. No. 5,830,910 to Mattson describes novel therapeutic uses of certain compounds to protect nerve cells from injury and death. The compounds include cytochalasin D and related analogs, and cytochalasin E and related analogs.

Palmae or Arecaceae, the Palm Family, is a family of flowering plants belonging to the monocot order, Arecales. There are roughly 202 currently known genera with around 2600 species, most of which are restricted to tropical, subtropical, and possibly warm temperate climates. Most palms are distinguished by their large, compound, evergreen leaves arranged at the top of an unbranched stem. However, many palms are exceptions to this statement, and palms in fact exhibit an enormous diversity in physical characteristics. As well as being morphologically diverse, palms also inhabit nearly every type of habitat within their range, from rainforests to deserts. Palms are one of the most well-known and extensively cultivated plant families. They have had an important role to humans throughout much of history. Many common products and foods are derived from palms, and palms are also widely used in landscaping for their exotic appearance. However, there is no indication in publications regarding palms possessing neuroprotective properties

In view of the above, it is advantageous to provide an extract with neuroprotective property prepared from a plant of the family Palmae.

### SUMMARY OF THE INVENTION

The present invention provides an extract having neuroprotective property prepared from a plant of the family Palmae, which is capable in preserving neurons against damage caused by oxidation, ageing and/or nitric oxide deficiency. More specifically, the extract is capable of protecting brain cells without affecting the growth of normal cells.

In an aspect of the present invention, there is provided a novel use of an extract prepared from a plant of the family Palmae in the manufacture of a pharmaceutical preparation for protecting against neuron damage.

In another aspect of the present invention, there is provided a pharmaceutical preparation comprising an extract obtained from a plant of the family Palmae as an active ingredient in a therapeutically effective amount to protect against neuron damage and a pharmaceutically acceptable carrier, diluents or excipient.

The pharmaceutical preparation of the invention can be formulated into therapeutic dosage forms such as tablets, capsules, liquid orals, sterile injections, lotions, aqueous or oily solutions or suspensions and the like. The preparation may be administered in by known techniques, such as oral and parenteral administration (including subcutaneous injection, intravenous or intramuscular technique), in dosage unit formulations containing conventional non-toxic pharmaceutically acceptable carriers, diluents, or excipient. The extract, as it is in the preparation, may be a liquid or a powder.

The extract according to the present invention may also be incorporated into comestibles, for example instant tea preparation, by methods known to a person skilled in the art.

In another aspect of the present invention, there is provided a method for the preparation of an extract of a plant of the family Palmae, which includes pre-treating palm leaves, subjecting the pre-treated palm leaves to extraction using a first polar extraction solvent comprising an alcohol, with or without a co-solvent; and concentrating the extraction solvent or solvents to form a powdery preparation or paste after removal of the pre-treated palm leaves.

### BRIEF DESCRIPTION OF THE DRAWINGS

The invention will now be illustrated, but not limited, by the following description and examples, and with reference to the accompanying drawings of which:

FIGS. 1a to 1c are graphs showing the effects of palm leaves extract, in which FIG. 1a shows the effect on hippocampal CA1 pyramidal cells, FIG. 1b shows the effect on hippocampal CA3 pyramidal cells and FIG. 1c shows the effect on hippocampal dentate gyrus (DG) granule cells in normal and nitric oxide (NO) deficient rats; and

FIGS. 2a to 2d are graphs showing the effects of palm leaves extract, in which FIG. 2a shows the effect on brain superoxide dismutase (SOD) activities, FIG. 2b shows the effect on brain glutathione peroxidase (GPx) expressions, FIG. 2c shows the effect on brain catalase expressions and FIG. 2d shows the effect on brain malondialdehyde (MDA) concentrations in normal and nitric oxide (NO) deficient rats.

### DETAILED DESCRIPTION OF THE INVENTION

A person skilled in the art will readily appreciate that the present invention is well adapted to carry out the objects and obtain the ends and advantages mentioned, as well as those inherent therein. The embodiment describes herein is not intended as limitations on the scope of the invention.

The term “therapeutically effective amount” used herein throughout the specification refers to the amount of the active ingredient, the extract, to be administered orally to the subject to trigger the desired effect without or causing minimal toxic adverse effect against the subject. One skilled in the art should know that the effective amount can vary from one individual to another due to the external factors such as age, sex, diseased state, races, body weight, formulation of the extract, availability of other active ingredients in the formulation and so on.

It is important to note that the extract used in the disclosed method in this embodiment is derived from the plant species of Palmae or Arecaceae family. The extracts obtained from the above-mentioned plant species are suitable to be incorporated into edible or topical products, or as capsules, ointments, lotions and tablets.

The desired compounds to be extracted from the extract of the invention are mainly constituted of, but not limited to, biophenols, proteins, lipids, saccharides, minerals and small peptides. Due to polarity of these compounds, the polar solvent such as alcohol is found to be effective in extracting these desired compounds from the plant matrix.

Another embodiment of the present invention involves a pharmaceutical and comestible preparation with neuroprotective property comprising solvent extracts derived from the leaves of Palmae family using an appropriate extraction solvent.

The pharmaceutical preparation of the invention can be formulated into therapeutic dosage forms such as tablets, capsules, liquid orals, sterile injections, aqueous or oily solutions or suspensions and the like. The preparation may be administered in by known techniques, such as oral and parenteral administration (including subcutaneous injection, intravenous or intramuscular technique), in dosage unit formulations containing conventional non-toxic pharmaceutically acceptable carriers, diluents or excipient. The extract of the leaves of Palmae family, as it is in the preparation, may be a liquid or a powder.

As used herein, the term pharmaceutically acceptable carrier means a non-toxic, inert solid, semi-solid or liquid filler, diluent, encapsulating material or formulation auxiliary of any type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cotton seed oil, safflower oil, sesame oil, olive oil, corn oil, and soybean oil; glycols, such as propylene glycol; esters, such as ethyl oleate and ethyl laurate; agar; buffering agents such as magnesium hydroxide and aluminum hydroxide; alginic acid; pyrogen-free water; isotonic saline; Ringer's is solution; ethyl alcohol; and phosphate buffer solutions, as well as other non-toxic compatible lubricants such as sodium laryl sulfate and magnesium stearate, as well as coloring agents, releasing agents, coating agents, sweetening, flavoring and perfuming agents; preservatives and antioxidants can also be present in the composition, according to the judgment of the formulator.

The pharmaceutical and comestible preparation of the invention may be prepared by mixing the various components of the preparation using conventional methods. The preferred composition of the preparation may be prepared according to the constituent ranges set forth herein in Table 1.

In an embodiment, the usual dose or therapeutically effective amount of the extract varies from about 10 to 400 mg/kg of body weight of the patient per day. More preferably the usual dose or therapeutically effective amount of the extract is in the range of from about 10 to 200 mg/kg of body weight of the patient administered in equal portions twice a day or thrice a day.

The comestibles mentioned herein can be any common daily consumed processed food such as bread, noodles, confections, chocolates, beverages (for example instant tea preparation), and the like. One skilled in the art shall appreciate the fact that the aforesaid extract can be incorporated into the processed comestibles, capsules, tablets or topical medicine during the course of processing. Therefore, any modification thereon shall not depart from the scope of the present invention.

As set forth in the above description, the pharmaceutical preparation with neuroprotective property comprises alcoholic or aqueous extract or their combinations from leaves of Palm family. Preferably, the plant is any one or combination of the plant species of, Elaeis guineensis, Elaeis oleifera, Phoenix dactylifera and Cocos nucifera. The inventors of the present invention found that the alcohol extract derived from the aforementioned species possesses both acceptable taste that confers the derived extract to be comfortably incorporated with the comestibles product, capsules, tablets or topical medicine with minimal additional refining process.

According to the preferred embodiment, the extract to be incorporated into the comestibles and medicine can be acquired from any known method not limited only to the foregoing disclosed method. Following another embodiment, the extract is prepared in a concentrated form, preferably paste or powdery form which enables the extract to be incorporated in various formulations of the comestibles, capsules, tablets or topical products.

In line with the preferred embodiment, the extract shall be the plant metabolites which are susceptible to an extraction solvent. The compounds and small peptides with the neuroprotective properties are those metabolites in the alcoholic extracts. Therefore, the alcoholic extracts of leaves of Palmae family is preferably derived from the extraction solvent of water, alcohol, acetone, chloroform, liquid CO₂ and any combination thereof.

In view of the prominent property of promoting neuroprotection by the extracts in a subject, further embodiment of the present invention includes a method comprising the step of administrating orally or topically to the subject an effective amount of an extract derived from the leaves of Palmae family.

The following examples are intended to further illustrate the invention, without any intent for the invention to be limited to the specific embodiments described therein.

### Example 1

Vegetative parts of the palm leaves were collected, cleaned, washed and cut into small pieces and oven-dried at 40° C. overnight. The dried material was ground using a blender and extracted three times with alcohol (1:10 v/v) and three times with hot or warm water or with mixtures of water, acetone, chloroform and alcohol.

Other solvents may be used as a medium for the extraction. This extraction process is designed to separate soluble compounds by diffusion from a solid matrix (i.e. plant tissue) using a liquid matrix (solvent). Alcohol, water, chloroform and acetone can be used to produce a desired yield in extracting the active components. The extraction was performed in a few minutes. The pooled extracts were vacuum-dried at 40° C. and stored until further use. The amount of extract obtained from the vegetative dried materials ranged from 1 to 40% by weight.

### Example 2

### Neuroprotective Activities of Palm Leaves Extract

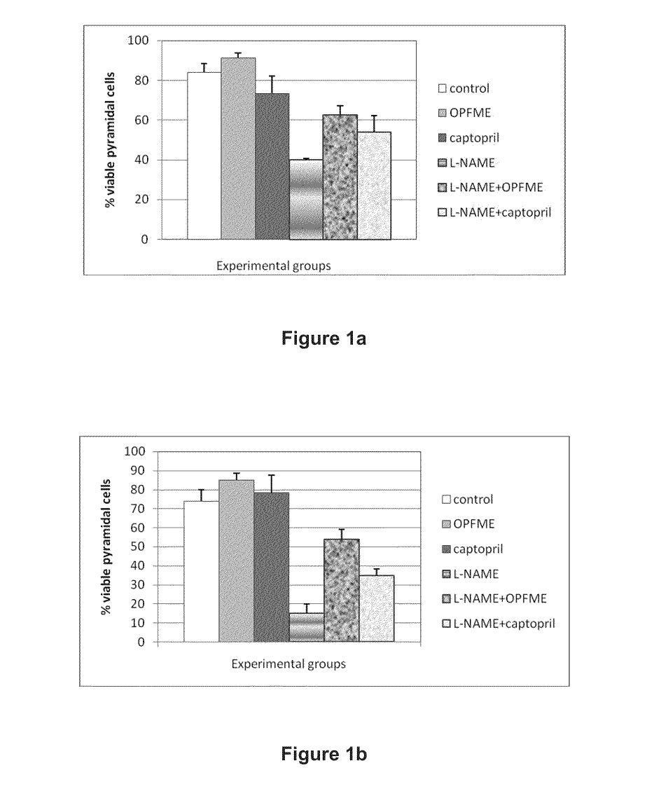

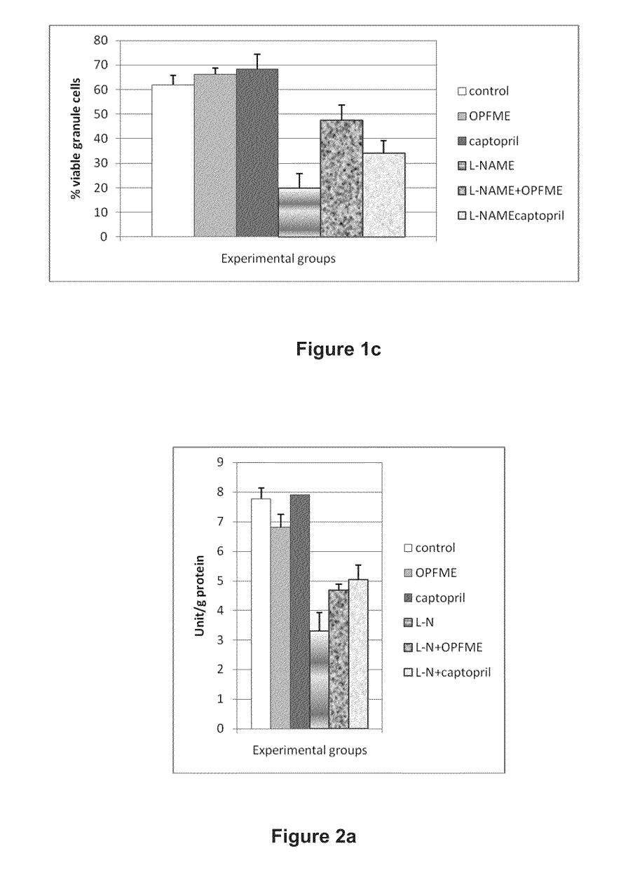

Neuroprotective activities of polyphenol-rich palm leaves extract and captopril were evaluated in normal and nitric oxide (NO) deficient rats by assessing neuron viability of three subfields in the hippocampus; CA1. CA3 and DG. L-NAME administration significantly reduced the number of viable pyramidal cells in CA1 to 40% (see FIG. 1a) and treatment with palm leaves extract in these rats significantly attenuated neurodegeneration, to values of 62% (p=0.000). Captopril showed partial neuroprotection in this region as the viable pyramidal cell count was lower than that of palm leaves extract. There was no dramatic neuron loss in all normal rats and treatment with palm leaves extract or captopril caused no significant change in viable pyramidal cell count compared to control, although in captopril group viability was slightly less.

The CA3 region was observed to be more affected by L-NAME than CA1 region as 85% of the pyramidal cells were severely injured, leaving only 15% viability (see FIG. 1b). Palm leaves extract or captopril treatment significantly protected CA3 pyramidal cells and maintained viability at 54% and 35% respectively (p=0.000). No significant difference in viability was observed between all 3 groups of normal rats.

Similarly, neurodegeneration in dentate gyrus (DG) of nitric oxide (NO) deficient rats is also extensive, leaving only 20% viable granule cells (see FIG. 1c) and again, neuroprotective activity of palm leaves extract or captopril significantly protected 48% and 34% neurons from degeneration (p=0.000), which are similar to those of CA3. The viability values of all 3 normal groups were similar.

The 12-week L-NAME administration to cause nitric oxide (NO) deficiency showed a dramatic reduction in viable neurons in the CA1. CA3 and DG of the hippocampus, and that co-administration with palm leaves extract attenuated this effect more effectively than with captopril. The pronounced loss of neurons with L-NAME administration caused the impairment in acquisition, but not retention, of spatial memory in the radial-arm maze task and the Morris water maze task. The neuroprotective effect of palm leaves extract was not significant in normal adult rats where loss of neurons have not occurred. The normal neuron count in palm leaves extract treated normal rats also reflects the absence of neurotoxicity.

### Example 3

### Antioxidative Properties of Palm Leaves Extract

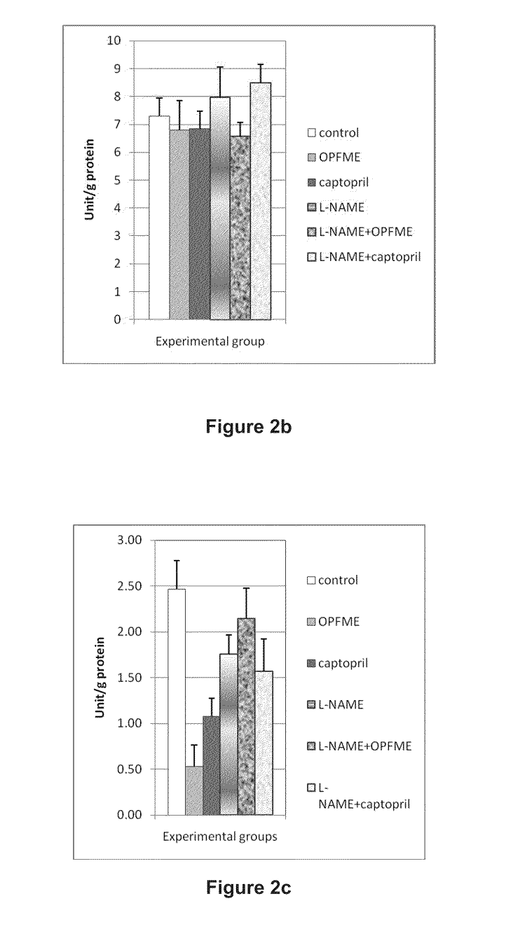

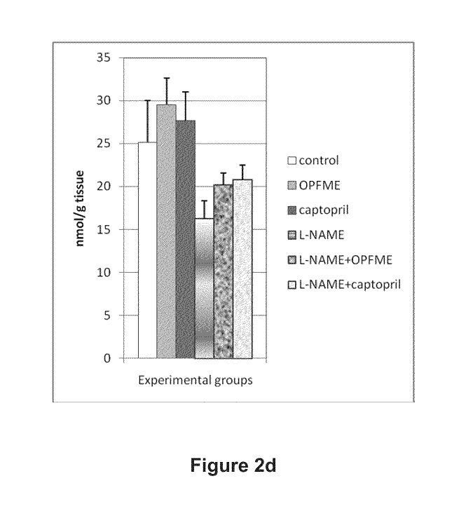

FIG. 2a shows L-NAME administration decreased superoxide dismutase (SOD) activity but this effect was attenuated significantly by palm leaves extract or captopril treatment (p=0,000). Palm leaves extract and captopril both increased superoxide dismutase (SOD) activities by 42% and 51% respectively when compared with L-NAME treated animals. All 3 groups of normal brain rats have similar superoxide dismutase (SOD) activities. Glutathione peroxidase (GPx) activity was not affected by the compromised nitric oxide (NO) environment as there was insignificant difference between all normal and all nitric oxide (NO) deficient groups (see FIG. 2b). FIG. 2c shows that catalase activity is depleted significantly in palm leaves extract or captopril treated normal rat group compared to control (p=0.033). In all nitric oxide (NO) deficient groups, catalase activities were slightly lowered without any statistical significance when compared to control. FIG. 2d shows that all nitric oxide (NO) deficient groups had brain malondialdehyde (MDA) level lower than normal groups and this reduction was significant in the untreated nitric oxide (NO) deficient group compared to control (p=0044). In normal rats, the malondialdehyde (MDA) level was similar in all three groups indicating that palm leaves extract did not attenuate malondialdehyde (MDA) production.

In all L-NAME administered rats the significant reduction in superoxide dismutase (SOD) activity was accompanied by a decrease in malondialdehyde (MDA) indicating that by inhibiting nitric oxide (NO) synthesis, the formation of lipid peroxides was also retarded. The diminishing nitric oxide (NO) in the brain may hinder the formation of peroxynitrite radical, which is well known to be more reactive than nitric oxide (NO) or superoxide. Normal brain aging is associated with elevated levels of neuro-inflammation, which is also intimately linked to enhanced reactive oxygen species (ROS) production.

The normal rats in this study had higher levels of malondialdehyde (MDA), superoxide dismutase (SOD) and catalase activities, which is the opposite effect observed in nitric oxide (NO) deficient rats, indicating that normal brain aging process could indeed be producing more nitric oxide (NO), which could lead to the formation of peroxynitrite and eventually causing more lipid peroxidation and increased inducible nitric oxide synthase (iNOS) expression in the hypothalamus and other brain regions of aged rats. The enhanced antioxidant defense observed was most likely counteracting the high rate of lipid peroxidation, eliminating oxidative stress and minimizing oxidative neuronal injury. The relatively young animal control group, despite having higher rate of lipid peroxidation than L-NAME group, still maintained an acceptably high viable neuron count. Even though a significant reduction in lipid peroxidation was evident in the untreated L-NAME rat brain, the number of viable neurons was considerably low, suggesting that the extent of nitric oxide synthase (NOS) inhibition, particularly the nitric oxide synthase from neurons (nNOS) could result in a nitric oxide (NO) level less than the physiological requirement for survival of the neurons.

The major neurotransmitter in the pathways connecting hippocampal dentate gyrus (DG) granule cells to CA3 and to CA1 is glutamate. There is a growing evidence to suggest that glutamate regulates the release of dopamine in the central nervous system (CNS). Palm leaves extract and captopril may have attenuated the neurodegeneration by increasing the availability of nitric oxide (NO). The partial neuroprotective effect of captopril could be due to the inhibition of angiotensin converting enzyme (ACE) which results in retardation of NADPH oxidase activity, a well-known source of reactive oxygen species (ROS). The reduced reactive oxygen species (ROS) level enhances the availability of residual nitric oxide (NO). The elevated superoxide dismutase (SOD) activities in palm leaves extract and captopril groups could also partially be responsible for the neuroprotective effect. By promoting endogenous antioxidant defense, palm leaves extract or captopril may prevent residual nitric oxide (NO) from oxidative degradation, enabling normal neuron function. Aged SOD-1 (Cu/Zn SOD) or aged EC-SOD (extracellular SOD) over-expressing mice reportedly improved hippocampal function compared to that of aged wild-type littermates, and aged EC-SOD transgenic mice are protected against a decrease in age-dependent impairments in spatial memory. This is in contrast to the antioxidant effect of palm leaves extract observed in all normal rat brains, and could probably be due to the different physiological environment of the two rat models. In a normal physiological environment palm leaves extract merely maintains the antioxidant defense but in pathological conditions, such as nitric oxide (NO) insufficiency which increases the risk of neuronal injury, it also improves the endogenous antioxidant enzyme defense which could minimize injury.

In normal rats, palm leaves extract or captopril dramatically decreased catalase activity whilst maintaining superoxide dismutase (SOD) activity and malondialdehyde (MDA) level. This indicate that both palm leaves extract and captopril effectively diminished oxidative stress by acting as an antioxidant and as a result lessened the requirement for selected endogenous antioxidant defense. Palm leaves extract probably inhibited lipid peroxidation, by reducing the level of hydrogen peroxide (H₂O₂), which may also cause oxidative injury. In vitro evidence suggested that catalase could be induced in cultured vascular cells in the presence of either lipid peroxides or H₂O₂, and vice versa, when the peroxide level decreases, catalase activity is expected to be lower.

### Example 4

### Composition Palm Leave Extract

Palm leaves extract of the present invention was combined with tocotrienol and magnesium stearate to form a composition with the amounts listed below in Table 1. The composition was then made into the form of a capsule.

TABLE 1
Content of Each Capsule

| Ingredient | mg/capsule |
|---|---|
| Palm leaves extract | 450.0 |
| Tocotrienol rich palm oil fraction | 1.0 |
| Magnesium stearate | 3.0 |
| Total fill weight | 454.0 |

While the present invention has been described with reference to several particular implementations, those skilled in the art will recognize that many changes may be made hereto without departing from the scope of the present invention.

1-6. (canceled)

7. A pharmaceutical preparation comprising an extract obtained from a material of a plant of the family Palmae as an active ingredient in a therapeutically effective amount to protect against neuron damage, wherein the weight percentage of the plant extract in the pharmaceutical preparation is up to 100%.

8. A pharmaceutical preparation according to claim 7 further comprising a pharmaceutically acceptable carrier, diluents or excipient.

9. A pharmaceutical preparation according to claim 7, wherein the therapeutically effective amount of the extract comprises dosage in the range of from 10 to 400 mg/kg of body weight of the patient.

10. A pharmaceutical preparation according to claim 9, wherein the therapeutically effective amount of the extract comprises dosage in the range of from 10 to 200 mg/kg of body weight of the patient.

11. A pharmaceutical preparation according to claim 7, wherein the Palmae family is any one or in combination of Elaeis guineensis, Elaeis oleifera, Phoenix dactylifera and Cocos nucifera.

12. A pharmaceutical preparation according to claim 7, wherein the plant material includes palm leaves.

13. A pharmaceutical preparation according to claim 7, wherein the extract is a solvent extract.

14. A comestible preparation comprising an extract obtained from a material of a plant of the family Palmae as an active ingredient in a therapeutically effective amount to protect against neuron damage, wherein the weight percentage of the plant extract in the comestible preparation is up to 100%.

15. A comestible preparation according to claim 14, wherein the therapeutically effective amount of the extract comprises dosage in the range of from 10 to 400 mg/kg of body weight of the patient.

16. A comestible preparation according to claim 15, wherein the therapeutically effective amount of the extract comprises dosage in the range of from 10 to 200 mg/kg of body weight of the patient.

17. A comestible preparation according to claim 14, wherein the Palmae family is any one or in combination of Elaeis guineensis, Elaeis oleifera, Phoenix dactylifera and Cocos nucifera.

18. A comestible preparation according to claim 14, wherein the plant material includes palm leaves.

19. A comestible preparation according to claim 14, wherein the extract is a solvent extract.

20. A Method of using a plant of the family Palmae comprising the steps of
(i) extracting a material from the plant of the family Palmae and
(ii) preparing a pharmaceutical compound with said extracted material, and
(iii) protecting against neuron damage with said pharmaceutical compound.

21. The method of claim 20, wherein the weight percentage of the plant extract is from 1% to 40%.

22. The method of claim 20, wherein step (iii) comprises at least one of the following
(iii.a) alleviating oxidative stress
(iii.b) alleviating nitric oxide deficiency, or
(iii.c) alleviating aging.

23. The method of claim 20, wherein the family Palmae includes at least one of
Elaeis guineensis
Elaeis oleifera
Phoenix dactylifera, and
Cocos nucifera

24. The method of claim 20, wherein the plant material includes palm leaves.

25. The method of claim 20, wherein the plant extract is a solvent extract.
